(19) United States
(12) Patent Application Publication
(10) Pub. No.: US 2009/0004756 A1
(43) Pub. Date: Jan. 1, 2009

(54) METHODS FOR IDENTIFYING AGENTS THAT TARGET LEAKS IN RYANODINE RECEPTORS

(75) Inventor: Andrew R. Marks, Larchmont, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/121,446

(22) Filed: May 15, 2008

Related U.S. Application Data
(63) Continuation-in-part of application No. 11/088,123, filed on Mar. 23, 2005, now Pat. No. 7,393,652.
Application No. 11/088,123 is a division of application No. 10/763,498, filed on Jan. 22, 2004, now abandoned.
Application No. 10/763,498 is a continuation-in-part of application No. 10/680,988, filed on Oct. 7, 2003.
Application No. 10/680,988 is a continuation-in-part of application No. 10/608,723, filed on Jun. 26, 2003, now abandoned.
Application No. 10/608,723 is a continuation-in-part of application No. 10/288,606, filed on Nov. 5, 2002, now abandoned.
Application No. 10/288,606 is a continuation of application No. 09/568,474, filed on May 10, 2000, now Pat. No. 6,489,125.
Application No. 09/568,474 is a continuation-in-part of application No. 11/933,745, filed on Nov. 1, 2007.
Application No. 11/933,745 is a division of application No. 10/794,218, filed on Mar. 5, 2004, now Pat. No. 7,312,044.
Application No. 10/794,218 is a continuation-in-part of application No. 11/506,285, filed on Aug. 17, 2006.
Application No. 11/506,285 is a continuation-in-part of application No. 11/212,309, filed on Aug. 25, 2005.
(60) Provisional application No. 60/452,664, filed on Mar. 7, 2003.

(51) Int. Cl. G01N 33/566

(52) U.S. Cl. 436/501

(57) ABSTRACT

The present invention provides screening methods for identifying agents that enhance binding of a ryanodine receptor and its corresponding FKBP protein, such agents to be used in treating diseases associated with ryanodine receptors.

### CROSS-REFERENCE TO RELATED APPLICATIONS

This application is (1) a Continuation-in-Part of U.S. patent application Ser. No. 11/088,123, filed on Mar. 23, 2005, which is a divisional application of U.S. patent application Ser. No. 10/763,498, filed on Jan. 22, 2004, now abandoned, which is a Continuation-in-Part of U.S. patent application Ser. No. 10/680,988, filed on Oct. 7, 2003, which is a Continuation-in-Part of U.S. patent application Ser. No. 10/608,723, filed on Jun. 26, 2003, which is a Continuation-in-Part of patent application Ser. No. 10/288,606, filed on Nov. 5, 2002, which is a continuation of U.S. patent application Ser. No. 09/568,474, filed on May 10, 2000, now U.S. Pat. No. 6,489,125 B1; and (2) a Continuation-in-Part of U.S. patent application Ser. No. 11/933,745, filed Nov. 1, 2007, which is a continuation of U.S. patent application Ser. No. 10/794,218, filed Mar. 5, 2004, now U.S. Pat. No. 7,312,044 B2, which claims priority to U.S. provisional patent application No. 60/452,664, filed Mar. 7, 2003; and (3) a Continuation-in-Part of U.S. patent application Ser. No. 11/506,285 filed on Aug. 17, 2006 which is a Continuation-in-Part of U.S. patent application Ser. No. 11/212,309 filed on Aug. 25, 2005; the contents of each of which are hereby incorporated by reference thereto.

### STATEMENT OF GOVERNMENT INTEREST

This invention was made with government support under NIH Grant No. PO1 HL 67849-01. As such, the United States government has certain rights in this invention.

### BACKGROUND OF THE INVENTION

The sarcoplasmic reticulum (SR) is a structure in cells that functions, among other things, as a specialized intracellular calcium (Ca²⁺) store. Channels in the SR called ryanodine receptors (RyRs) open and close to regulate the release of Ca²⁺ from the SR into the intracellular cytoplasm of the cell. There are three types of RyRs, all of which are highly-related Ca²⁺ channels: RyR1, RyR2, and RyR3. RyR1 is found predominantly in the skeletal muscle as well as other tissues, RyR2 is found predominantly in the heart as well as other tissues, and RyR3 is found in the brain as well as other tissues. The RyR channels are formed by four RyR polypeptides in association with four FK506 binding proteins (FKBP proteins), specifically FKBP12 (calstabin1) and FKBP12.6 (calstabin2). FKBP12 binds to RyR1, FKBP12.6 binds to RyR2, and FKBP12 binds to RyR3. The FKBP proteins (calstabin1 and calstabin2) bind to the RyR channel (one molecule per RyR subunit), stabilize RyR-channel functioning, and facilitate coupled gating between neighboring RyR channels, thereby preventing abnormal activation of the channel during the channel's closed state. The binding of the FKBP proteins to the RyR channels is modulated by Protein Kinase A (PKA). In particular, PKA phosphorylation of RyR increases the open probability of the channel by dissociating FKBP protein from the channel complex. This, in turn, increases the sensitivity of RyR to Ca²⁺-dependent activation.

Ca²⁺ release from the SR in skeletal muscle and heart cells is a key physiological mechanism that controls muscle performance because increased concentration of Ca2+ in the intracellular cytoplasm causes contraction of the muscle.

Excitation-contraction (EC) coupling in skeletal muscles involves electrical depolarization of the plasma membrane in the transverse tubule (T-tubule), which activates voltage-gated L-type Ca²⁺ channels (LTCCs). LTCCs trigger Ca²⁺ release from the SR through physical interaction with RyR1. The resulting increase in cytoplasmic Ca²⁺ concentration induces actin-myosin interaction and muscle contraction. To enable relaxation, intracellular Ca²⁺ is pumped back into the SR via SR Ca²⁺-ATPase pumps (SERCAs), which are regulated by phospholamban (PLB) depending on the muscle fiber type.

In cardiac striated muscle, RyR2 is the major Ca²⁺-release channel required for EC coupling and muscle contraction. During EC coupling, depolarization of the cardiac-muscle cell membrane during phase zero of the action potential activates voltage-gated Ca²⁺ channels. Ca²⁺ influx through the open voltage-gated channels in turn initiates Ca²⁺ release from the SR via RyR2. This process is known as Ca²⁺-induced Ca²⁺ release. The RyR2-mediated, Ca²⁺-induced Ca²⁺ release then activates the contractile proteins in the cardiac cell, resulting in cardiac muscle contraction.

Phosphorylation of cardiac RyR2 by PKA is an important part of the “fight or flight” response that increases cardiac EC coupling gain by augmenting the amount of Ca²⁺ released for a given trigger. This signaling pathway provides a mechanism by which activation of the sympathetic nervous system, in response to stress, results in increased cardiac output. PKA phosphorylation of RyR2 increases the open probability of the channel by dissociating calstabin2 from the channel complex. This, in turn, increases the sensitivity of RyR2 to Ca²⁺-dependent activation.

PKA hyperphosphorylation of RyR2 has been proposed as a factor contributing to depressed contractile function and arrhythmogenesis in heart failure. Consistent with this hypothesis, PKA hyperphosphorylation of RyR2 in failing hearts has been demonstrated, in vivo, both in animal models and in patients with heart failure undergoing cardiac transplantation.

In failing hearts, the hyperphosphorylation of RyR2 by PKA induces the dissociation of calstabin2 from the RyR2 channel. This causes marked changes in the biophysical properties of the RyR2 channel, including increased open probability due to an increased sensitivity to Ca²⁺-dependent activation; destabilization of the channel, resulting in subconductance states; and impaired coupled gating of the channels, resulting in defective EC coupling and cardiac dysfunction. Thus, PKA-hyperphosphorylated RyR2 is very sensitive to low-level Ca²⁺ stimulation, and this manifests itself as a diastolic SR Ca²⁺ leak through PKA hyperphosphorylated RyR2 channels.

The maladaptive response to stress in heart failure results in depletion of calstabin2 from the channel macromolecular complex. This leads to a shift to the left in the sensitivity of RyR2 to Ca²⁺-induced Ca²⁺ release, resulting in channels that are more active at low-to-moderate Ca²⁺ concentrations. Over time, the increased “leak” through RyR2 results in resetting of the SR Ca²⁺ content to a lower level, which in turn reduces EC coupling gain and contributes to impaired systolic contractility.

Additionally, a subpopulation of RyR2 that are particularly “leaky” can release SR Ca²⁺ during the resting phase of the cardiac cycle, diastole. This results in depolarizations of the cardiomyocyte membrane known as delayed after-depolarizations (DADs), which are known to trigger fatal ventricular cardiac arrhythmias.

U.S. patent application Ser. Nos. 10/608,723, 10/288,606 and 09/568,474 (now U.S. Pat. No. 6,489,125 B1) disclose a novel mechanism underlying heart failure and cardiac arrhythmias and methods of treatment thereof. In particular, these patent applications show that exercise-induced cardiac arrhythmias and sudden cardiac death, as well as heart failure result from a reduced affinity between cardiac RyR2 and its binding protein FKBP12.6, and that some 1,4-benzothiazepine compounds modulate the function of RyR2 calcium-ion channels by increasing FKBP12.6 binding to RyR2. Additionally, U.S. Pat. No. 7,312,044 discloses that dissociation of FKBP12 from RyR1 channels may be responsible for skeletal muscular disorders.

There is a need to identify new agents for treating disorders and diseases associated with RyRs, including skeletal muscular, cardiac and cognitive disorders and diseases. More particularly, a need remains to identify new agents that can be used to treat RyR associated disorders by, for example, repairing the leak in RyR channels, and enhancing binding of calstabin proteins to PKA-phosphorylated RyRs, and to mutant RyRs that otherwise have reduced affinity for, or do not bind to, calstabins. This invention now provides solutions to these needs.

### SUMMARY OF THE INVENTION

The present invention provides methods for identifying agents that directly enhance binding of an FKBP protein to its corresponding ryanodine receptor (RyR). In one embodiment, the method comprises:
(a) exposing the RyR to the FKBP protein, in the presence or absence of a candidate chemical compound in vitro;
(b) determining the amount of the FKBP protein bound to the RyR in the presence and/or absence of the compound, wherein an increased amount of the FKBP protein bound to the RyR in the presence of the compound indicates that the compound directly enhances binding of the FKBP protein to the RyR, or determining the amount of the RyR bound to the FKBP protein in the presence and/or absence of the compound, wherein an increased amount of RyR bound to the FKBP protein in the presence of the compound indicates that the compound directly enhances binding of the FKBP protein to the RyR; thereby
(c) identifying the compound as a compound that directly enhances binding of the FKBP protein to the RyR.

In one embodiment of the present method, the FKBP protein is immobilized onto a solid phase. Alternatively, the RyR may be immobilized onto a solid phase.

In certain embodiments, step (a) of the present method is conducted by:
(i) immobilizing one of the FKBP protein or the RyR onto a solid phase;
(ii) contacting the RyR with the FKBP protein immobilized onto a solid phase or the FKBP protein with the RyR immobilized onto a solid phase; and
(iii) incubating the RyR with the FKBP protein immobilized onto a solid phase in the presence or absence of a candidate chemical compound in vitro, or incubating the FKBP protein with the RyR immobilized onto a solid phase in the presence or absence of a candidate chemical compound in vitro.

The solid phase is preferably a plate, a bead, a column, or a combination thereof. To facilitate high throughput screening, the solid phase may be a multi-well plate. In this embodiment, the amount of the RyR bound to the FKBP protein in each well can be determined by an automated plate reader. In certain embodiments, it is possible to configure the multi-well plate so that a pre-determined number of wells on the plate contain no candidate chemical compound, and wherein other wells on the plate each contain one candidate chemical compound.

In certain embodiments, the RyR in the above methods of the present invention is PKA-phosphorylated, for example but not limited to phosphorylation as a result of disease state, or in vitro PKA phosphorylation. In other embodiments, the RyR in the above methods of the present invention is a RyR mutant that has low affinity for its corresponding FKBP protein, such as RyR2-S2808D and RyR1-S2844D.

In certain embodiments, the RyR is radio-labeled with ³H-ryanodine, ³⁵S, ³²P, ³H, ¹⁴C, ¹²⁵I, or another radioactive isotope(s), so as to provide more quantifiable read-out. In other embodiments, FKBP protein is labeled with ³⁵S, ³²P, ³H, ¹⁴C, ¹²⁵I, or another radioactive isotope(s). In another embodiment of the present method, the RyR or FKBP protein is labeled with a fluorescent label.

In certain embodiments, the RyR is RyR1 and the FKBP protein is FKBP12, or the RyR is RyR2 and the FKBP protein is FKBP12.6.

In yet another embodiment, the enhanced binding of the RyR and the FKBP protein is detected by using an RyR-binding agent. In this embodiment, the RyR binding agent is an anti-RyR antibody.

### DETAILED DESCRIPTION OF THE INVENTION

As used herein, the term “FKBP protein” includes FKBP12 (calstabin1) and FKBP12.6 (calstabin2) as well as other FK506 binding proteins and their analogues. Unless otherwise indicated herein, “protein” shall include a protein, protein domain, polypeptide, or peptide, and any fragment thereof. As an example of an “FKBP analogue”, an “FKBP 12.6 analogue” is a functional variant of the FKBP12.6 protein, having FKBP12.6 biological activity, that has 60% or greater amino-acid-sequence homology with the FKBP12.6 protein. As used herein, the term “FKBP12.6 analogue.” includes FKBP12 and other FK506 binding proteins. As further used herein, the term “FKBP 12.6 biological activity” refers to the activity of a protein or peptide that demonstrates an ability to associate physically with, or bind with, unphosphorylated or non-hyperphosphorylated RyR2 (i.e., binding of approximately two fold, or, more preferably, approximately five fold, above the background binding of a negative control), under the conditions of the assays described herein, although affinity may be different from that of FKBP12.6.

As used herein, the term “RyR” includes RyR1, RyR2, and RyR3 as well as their analogues. As an example of an “RyR analogue”, an “RyR2 analogue” is a functional variant of the RyR2 protein, having RyR2 biological activity, that has 60% or greater amino-acid-sequence homology with the RyR2 protein. As used herein, the term “RyR2 analogue” includes RyR1—the skeletal-muscle isoform of RyR2, and RyR3—the central nervous sytem isoform of RyR2. As further used herein, the term “RyR2 biological activity” refers to the activity of a protein or peptide that demonstrates an ability to associate physically with, or bind with, the FKBP 12.6 protein (i.e., binding of approximately two fold, or, more preferably, approximately five fold, above the background binding of a negative control), under the conditions of the assays described herein, although affinity may be different from that of RyR2.

The cardiac ryanodine receptor, RyR2, is a protein complex comprising four 565,000-dalton RyR2 proteins in association with four 12,000-dalton FKBP12.6 proteins. FK506 binding proteins (FKBPs) are cis-trans peptidyl-prolyl isomerases that are widely expressed, and serve a variety of cellular functions. FKBP12.6 protein is tightly bound to, and regulates the function of, RyR2. FKBP12.6 binds to the RyR2 channel, one molecule per RyR2 subunit, stabilizes RyR2-channel function, and facilitates coupled gating between neighboring RyR2channels, thereby preventing aberrant activation of the channel during the resting phase of the cardiac cycle. Accordingly, as used herein, the term “RyR2-bound FKBP12.6” includes a molecule of an FKBP12.6 protein that is bound to a RyR2 protein subunit or a tetramer of FKBP 12.6 that is in association with a tetramer of RyR2.

As used herein, the phrases “an FKBP protein” and “its corresponding ryanodine receptor (RyR)” include any of the following: (i) FKBP 12 and RyR1; (2) FKBP 12.6 and RyR2; and (iii) FKBP 12 and RyR3.

As used herein, the phrases “RyR” and “its corresponding FKBP protein” include any of the following: (i) RyR1 and FKBP12; (2) RyR2 and FKBP12.6; and (iii) RyR3 and FKBP12.

The binding of calstabin1 (FKBP12) to RyR1 stabilizes the closed state of the RyR1 channel and facilitates coupled gating between neighboring channels (Brillantes, Ondrias et al. 1994; Marx, Ondrias et al. 1998). Mutation of RyR1 resulting in the loss of calstabin1 binding causes impaired ECC with reduced maximal voltage-gated SR Ca²⁺ release without affecting the SR Ca²⁺ store content (Avila, Lee et al. 2003). Genetic deletion of FKBP12 (calstabin1) in skeletal muscle-specific knock-out resulted in reduced voltage-gated SR Ca²⁺ release and increased L-type channel currents in isolated myotubes (Tang, Ingalls et al. 2004). These data indicate that reduced binding of calstabin1 (FKBP12) to RyR1 results in skeletal muscular diseases and disorders.

It has also been established that exercise-induced sudden cardiac death is associated with an increase in phosphorylation of RyR2 proteins (e.g., CPVT-associated RyR2mutant proteins) and a decrease in the level of RyR2-bound FKBP12.6. It is possible to use this mechanism to design effective drugs for preventing exercise-induced sudden cardiac death as well as other disease associated with RyRss. A candidate agent having the ability to limit or prevent a decrease in the level of RyR2-bound FKBP12.6 may, as a consequence of this limiting or preventive activity, have an effect on a RyR2-associated biological event, thereby preventing exercise-induced sudden cardiac death.

Additionally, deletion of RyR3 in knockout mice has been shown to impair forms of synaptic plasticity and spatial learning (Balschun et al., EMBO Journal (1999) 18, 5264-5273 “Deletion of the ryanodine receptor type 3 (RyR3) impairs forms of synaptic plasticity and spatial learning”). Kohda et al. found that Ca²⁺ release from ryanodine-sensitive Ca²⁺ stores is required for the induction of long term depression “LTD” in cultured cerebellar Purkinje cells. (Khoda et al. (1995) J Neurophysiol, 74,2184-2 188 “ca2+ release from ca2+ stores, particularly from ryanodinesensitive Ca²⁺ stores, is required for the induction of LTD in cultured cerebellar Purkinje cells.”) These studies suggest that reduced binding of calstabin1 (FKBP12) to RyR3 results in cognitive diseases and disorders.

Accordingly, the present invention further provides a method for identifying an agent or to identify compounds that affect the interaction between RyRs and calstabins by inhibiting dissociation of calstabin from RyR or by enhancing association of the two proteins, and such agent or compound could be useful in preventing exercise-induced sudden cardiac death as well as other disease associated with RyRs such as heart failure and skeletal muscle fatigue.

In one embodiment, the method comprises the steps of: (a) obtaining or generating a culture of cells containing RyR2; (b) contacting the cells with a candidate agent; (c) exposing the cells to one or more conditions known to increase phosphorylation of RyR2 in cells; and (d) determining if the agent limits or prevents a decrease in the level of RyR2-bound FKBP12.6 in the cells. In one embodiment, the RyR is RyR1 or RyR3, and the FKBP protein is FKBP12. In certain embodiments, the culture of cells is an isolated, or in vitro culture of cells.

As used herein, an “agent” may be a protein, polypeptide, peptide, nucleic acid (including DNA or RNA), antibody, Fab fragment, F(ab′)₂ fragment, molecule, compound, antibiotic, drug, and any combination(s) thereof. An agent that limits or prevents a decrease in the level of RyR2-bound FKBP12.6 may be either isolated or synthetic, and may be an agent reactive with (i.e., an agent that has affinity for, binds to, or is directed against) RyR2 and/or FKBP12.6. As further used herein, a cell “containing RyR2” is a cell (preferably, a cardiac muscle cell) in which RyR2, or a derivative, or homologue thereof, is expressed. In certain embodiments, RyR2, or a derivative, or homologue, thereof is naturally expressed or naturally occurs.

Conditions known to increase phosphorylation of RyR2 in cells include, without limitation, PKA.

In the method of the present invention, cells may be contacted with a candidate agent by any of the standard methods of effecting contact between drugs/agents and cells, including any modes of introduction and administration described herein. The level of RyR2-bound FKBP12.6, or RyR1-bound FKBP, or RyR3-bound FKBP in the cell may be measured or detected by known procedures, including any of the methods, molecular procedures, and assays known to one of skill in the art or described herein. In one embodiment of the present invention, the agent limits or prevents a decrease in the level of RyR2-bound FKBP 12.6 in the cells.

As disclosed herein, RyR2 has been implicated in a number of biological events in striated muscle cells. For example, it has been shown that RyR2 channels play an important role in EC coupling and contractility in cardiac muscle cells. Therefore, it is clear that preventive drugs designed to limit or prevent a decrease in the level of RyR2-bound FKBP12.6 in cells, particularly cardiac muscle cells, may be useful in the regulation of a number of RyR2-associated biological events, including EC coupling and contractility. Thus, once the candidate agent of the present invention has been screened, and has been determined to have a suitable limiting or preventive effect on decreasing levels of RyR2-bound FKBP 12.6, it may be evaluated for its effect on EC coupling and contractility in cells, particularly cardiac muscle cells. It is expected that the preventive agent of the present invention will be useful for preventing exercise-induced sudden cardiac death.

Accordingly, the method of the present invention may further comprise the steps of: (e) contacting the candidate agent with a culture of cells containing RyR2; and (f) determining if the agent has an effect on a RyR2-associated biological event in the cells. As used herein, an “RyR2-associated biological event” includes a biochemical or physiological process in which RyR2 levels or activity have been implicated. As disclosed herein, examples of RyR2-associated biological events include, without limitation, EC coupling and contractility in cardiac muscle cells. According to this method of the present invention, a candidate agent may be contacted with one or more cells (preferably, cardiac muscle cells) in vitro. For example, a culture of the cells may be incubated with a preparation containing the candidate agent. The candidate agent's effect on a RyR2-associated biological event then may be assessed by any biological assays or methods known in the art, including immunoblotting, single-channel recordings and any others disclosed herein.

The present invention is further directed to an agent identified by the above-described identification method, as well as a pharmaceutical composition comprising the agent and a pharmaceutically-acceptable carrier. The agent may be useful for preventing exercise-induced sudden cardiac death in a subject, and for treating or preventing other RyR2-associated conditions. As used herein, an “RyR2-associated condition” is a condition, disease, or disorder in which RyR2 level or activity has been implicated, and includes a RyR2-associated biological event. The RyR2-associated condition may be treated or prevented in the subject by administering to the subject an amount of the agent effective to treat or prevent the RyR2-associated condition in the subject. This amount may be readily determined by one skilled in the art. In one embodiment, the present invention provides a method for preventing exercise-induced sudden cardiac death in a subject, by administering the agent to the subject in an amount effective to prevent the exercise-induced sudden cardiac death in the subject.

The present invention also provides an in vivo method for identifying an agent for use in preventing exercise-induced sudden cardiac death. The method comprises the steps of: (a) obtaining or generating an animal containing RyR2; (b) administering a candidate agent to the animal; (c) exposing the animal to one or more conditions known to increase phosphorylation of RyR2 in cells; and (d) determining if the agent limits or prevents a decrease in the level of RyR2-bound FKBP12.6 in the animal. The method may further comprise the steps of: (e) administering the agent to an animal containing RyR2; and (f) determining if the agent has an effect on a RyR2-associated biological event in the animal. Also provided is an agent identified by this method; a pharmaceutical composition comprising this agent; and a method for preventing exercise-induced sudden cardiac death in a subject, by administering this agent to the subject in an amount effective to prevent the exercise-induced sudden cardiac death in the subject.

Compounds which block PKA activation would be expected to reduce the activation of the RyR2 channel, resulting in less release of calcium into the cell. Compounds that bind to the RyR2 channel at the FKBP12.6 binding site, but do not come off the channel when the channel is phosphorylated by PKA, would also be expected to decrease the activity of the channel in response to PKA activation or other triggers that activate the RyR2 channel. Such compounds would also result in less calcium release into the cell. In view of these findings, the present invention further provides additional assays for identifying agents that may be useful in preventing exercise-induced sudden cardiac death, in that they block or inhibit activation of RyR2.

By way of example, the diagnostic assays of the present invention may screen for the release of calcium into cells via the RyR2 channel, using calcium-sensitive fluorescent dyes (e.g., Fluo-3, Fura-2, and the like). Cells may be loaded with the fluorescent dye of choice, then stimulated with RyR2 activators to determine whether or not compounds added to the cell reduce the calcium-dependent fluorescent signal (Brillantes et al., Stabilization of calcium release channel (ryanodine receptor) function by FK506-binding protein. Cell, 77:513-23, 1994; Gillo et al., Calcium entry during induced differentiation in murine erythroleukemia cells. Blood, 81:783-92, 1993; Jayaraman et al., Regulation of the inositol 1,4,5-trisphosphate receptor by tyrosine phosphorylation. Science, 272:1492-94, 1996). Calcium-dependent fluorescent signals may be monitored with a photomultiplier tube, and analyzed with appropriate software, as previously described (Brillantes et al., Stabilization of calcium release channel (ryanodine receptor) function by FK506-binding protein. Cell, 77:513-23, 1994; Gillo et al., Calcium entry during induced differentiation in murine erythroleukemia cells. Blood, 81:783-92, 1993; Jayaraman et al., Regulation of the inositol 1,4,5-trisphosphate receptor by tyrosine phosphorylation. Science, 272:1492-94, 1996). This assay can easily be automated to screen large numbers of compounds using multi-well dishes.

To identify compounds that inhibit the PKA-dependent activation of RyR2-mediated intracellular calcium release, an assay may involve the expression of recombinant RyR2 channels in a heterologous expression system, such as Sf9, HEK293, or CHO cells (Brillantes et al., Stabilization of calcium release channel (ryanodine receptor) function by FK506-binding protein. Cell, 77:513-23, 1994). RyR2 could also be co-expressed with beta-adrenergic receptors. This would permit assessment of the effect of compounds on RyR2 activation, in response to addition of beta-adrenergic receptor agonists.

The level of PKA phosphorylation of RyR2 which correlates with the degree of heart failure may also be assayed, and then used to determine the efficacy of compounds designed to block the PKA phosphorylation of the RyR2 channel. Such an assay may be based on the use of antibodies that are specific for the RyR2 protein. For example, the RyR2-channel protein may be immunoprecipitated, and then back-phosphorylated with PKA and [gamma³²P]-ATP. The amount of radioactive [³²P] label that is transferred to the RyR2 protein may be then measured using a phosphorimager (Marx et al., PKA phosphorylation dissociates FKBP12.6 from the calcium release channel (ryanodine receptor): defective regulation in failing hearts. Cell, 101:365-76, 2000).

Another assay of the present invention involves use of a phosphoepitope-specific antibody that detects RyR2 that is PKA phosphorylated on Ser 2809. Immunoblotting with such an antibody can be used to assess efficacy of therapy for heart failure and cardiac arrhythmias. Additionally, RyR2 S2809A and RyR2 S2809D knock-in mice may be used to assess efficacy of therapy for heart failure and cardiac arrhythmias. Such mice further provide evidence that PKA hyperphosphorylation of RyR2 is a contributing factor in heart failure and cardiac arrhythmias, by showing that the RyR2 S2809A mutation inhibits heart failure and arrhythmias, and that the RyR2 S2809D mutation worsens heart failure and arrhythmias.

The present invention provides a method for identifying an agent that enhances binding of RyR2 and FKBP 12.6, comprising the steps of: (a) obtaining or generating a source of RyR2; (b) exposing the RyR2 to FKBP12.6, in the presence of a candidate agent; and (c) determining if the agent enhances the binding of RyR2 and FKBP12.6. In one embodiment, the RyR2 is PKA-phosphorylated. In another embodiment, the RyR2 is PKA-hyperphosphorylated. In yet another embodiment, the RyR2 is unphosphorylated.

In one embodiment, the RyR2 is immobilized to a solid phase, such as a plate or beads. To facilitate detection of RyR2-FKBP 12.6 binding, the FKBP 12.6 may be radio-labeled (e.g., with ³²S or other radio or fluorescent labels). Furthermore, enhanced binding of RyR2 and FKBP12.6 may be detected using an FKBP12.6-binding agent. In one embodiment, the FKBP12.6-binding agent is an anti-FKBP12.6 antibody.

In certain embodiments, the present invention provides novel assays for regular or high-throughput screening of biologically-active small molecules, based upon rebinding of an FKBP protein and its corresponding RyR, to identify compounds that affect the interaction between the FKBP protein and the RyR by inhibiting dissociation of the FKBP protein from the RyR or by enhancing association of the two proteins.

The method involves immobilizing an FKBP protein on a support (e.g., on a column or onto a plate using, for example glutathione coated plate or a glutathione-bead column and reacting with GST-calstabin), and adding labeled RyR in the presence or absence of a test compound. The amount of RyR bound to calstabin or the amount of RyR eluted from a column in the presence or absence of the test compound is monitored. Advantageously, the method preferably uses a RyR mutant that has low affinity for calstabin (e.g., RyR2-S2808D, RyR1-S2844D), or RyR that is PKA hyperphosphorylated either as a result of disease state or PKA phosphorylated in-vitro. Due to the low binding affinity between the RyR mutant (or PKA-phosphorylated RyR) and calstabin, the ability of the test compound to increase the affinity of the proteins can be easily measured because the increase in the binding affinity of the RyR mutant and calstabin in the presence of the test compound will be more prominent than that of wildtype RyR and calstabin. RyR can be radio-labeled with ³H-ryanodine, or by other methods. The method can also be adapted to high throughput read-outs such as in a multi-well plate.

In particular, the present invention provides an in vitro method for identifying a chemical compound that directly enhances binding of an FKBP protein to its corresponding ryanodine receptor (RyR), the method comprising:
(a) exposing the RyR to the FKBP protein, in the presence or absence of a candidate chemical compound in vitro;
(b) determining the amount of the FKBP protein bound to the RyR in the presence and absence of the compound, wherein an increased amount of the FKBP protein bound to the RyR in the presence of the compound indicates that the compound directly enhances binding of the FKBP protein to the RyR, or determining the amount of the RyR bound to the FKBP protein in the presence and absence of the compound, wherein an increased amount of the RyR bound to the FKBP protein in the presence of the compound indicates that the compound directly enhances binding of the FKBP protein to the RyR thereby
(c) identifying the compound as a compound that directly enhances binding of the FKBP protein to the RyR.

In one embodiment, the RyR is PKA-phosphorylated as a result of disease state or in vitro PKA phosphorylation. In another embodiment, the RyR is a RyR mutant that has low affinity for its corresponding FKBP protein. In a preferred embodiment, the RyR mutant is RyR2-S2808D. In another preferred embodiment, the RyR mutant is RyR1-S2844D.

In the method of the present invention, the FKBP protein is immobilized to a solid phase, such as a plate or beads.

The present invention further provides a high-throughput in vitro method for screening a plurality of compounds simultaneously to identify one or more chemical compounds that directly enhance binding of an FKBP protein to its corresponding RyR, the method comprising:
(a) immobilizing the FKBP protein onto a solid support, for example but not limited to structure;
(b) contacting the RyR with FKBP protein immobilized onto the structure;
(c) incubating the structure with the RyR, in the presence or absence of each candidate chemical compound in vitro;
(d) determining the amount of the FKBP protein bound to the RyR in the presence and absence of each candidate compound, wherein an increased amount of the FKBP protein bound to the RyR in the presence of a candidate compound indicates that the candidate compound directly enhances binding of the FKBP protein to the RyR; thereby
(e) identifying one or more candidate compounds that directly enhance binding of the FKBP protein to the RyR.

In one embodiment, the FKBP protein is a wild-type FKBP protein. In another embodiment, the FKBP protein is a fusion protein, such as GST-FKBP. Immobilizing the FKBP protein onto the structure is accomplished using standard procedures known in the art.

In another embodiment, the structure is a multi-well plate, such as a 96-well plate coated with glutathione. In one preferred embodiment, a pre-determined number (e.g. n=1-3) of wells on the multi-well plate contain no candidate chemical compound so that the binding of the RyR and the FKBP protein in the absence of any candidate chemical compound can be measured. The other wells of the same multi-well plate each contains one candidate chemical compound for measuring the binding of the RyR and the FKBP protein in the presence of said candidate compound. In one embodiment, the amount of the RyR bound to the FKBP protein in each well is determined by an automated plate reader.

In one embodiment, the RyR in the present high-throughput in vitro screen method is PKA-phosphorylated as a result of disease state or in vitro PKA phosphorylation. In another embodiment, the RyR is a RyR mutant that has low affinity for its corresponding FKBP protein. In a preferred embodiment, the RyR mutant is RyR2-S2808D. In another preferred embodiment, the RyR mutant is RyR1-S2844D.

To facilitate detection of enhanced binding of the RyR and the FKBP protein, the RyR and/or FKBP protein may be radio-labeled. Labeling of RyR is accomplished using one of a variety of different radioactive labels known in the art. The radioactive label is, for example, a radioisotope that emits detectable radiation including, without limitation, ³H, ³⁵S, ³²P, ¹⁴C or ¹²⁵I. In one embodiment, the RyR is radio-labeled with ³H-ryanodine. Radioactivity emitted by the radioisotope can be detected by techniques well known in the art.

The RyR and/or FKBP protein may also be labeled with a fluorescent label. A fluorescent labeling group can be selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof. For further information on fluorescent label moieties and fluorescence techniques, see, e.g., Handbook of Fluorescent Probes and Research Chemicals, by Richard P. Haughland, Sixth Edition, Molecular Probes, (1996), which is hereby incorporated by reference in its entirety. One of skill in the art can readily select a suitable labeling group, and conjugate such a labeling group to the RyR or FKBP protein, without undue experimentation.

Furthermore, enhanced binding of the RyR and the FKBP protein may be detected using an RyR-binding agent. In one embodiment, the RyR-binding agent is an anti-RyR antibody. In another embodiment, enhanced binding of the RyR and the FKBP protein may be detected using an FKBP-binding agent. In one embodiment, the FKBP-binding agent is an anti-FKBP antibody.

In one embodiment, RyR2 may be loaded onto a FKBP12.6-coated plate (or RyR1 may be loaded onto a FKBP12-coated plate), and incubated with candidate chemical compounds such as 1,4-benzothiazepene compounds at various concentrations (10-1,000 nM) for 30 min. Thereafter, the plate may be washed to remove the unbound RyR2 or RyR1, and then incubated with anti-RyR2 or anti-RyR1 antibody (e.g., for 30 min). The plate may be washed again to remove unbound anti-RyR antibody, and then treated with florescent-labeled secondary antibody. The plate may be read by an automatic fluorescent plate reader for binding activity.

Alternatively, RyR1 or RyR2 may be PKA-phosphorylated in the presence of ³²P-ATP. Radioactive PKA-phosphorylated RyR1 or RyR2 maybe loaded onto an FKBP 12.6-coated or FKBP 12-coated, 96-well plate, in the presence of candidate chemical compounds such as 1,4-benzothiazepene compounds at various concentrations (10-100 nM) for 30 min. The plate may be washed to remove the unbound radiolabeled RyR1 or RyR2, and then read by an automatic plate reader.

In another embodiment, RyR1 or RyR2 is coated to the plate, and incubated with ³⁵S-labeled FKBP12.6 or ³⁵S-labeled FKBP12 in the presence of candidate chemical compounds such as 1,4-benzothiazepene compounds.

In one embodiment, the candidate chemical compounds are 1,4-benzothiazepine compounds such as those disclosed in co-pending U.S. application Ser. No. 11/212,309.

The methods of the invention have been applied to screen compounds from the genus described herein and compounds such as S4, S7, S20, S24-27 and S36 were identified as preventing dissociation of FKBP12.6 from RyR2 (see Example 13 and FIG. 12 of co-pending U.S. application Ser. No. 10/809,089). In addition to the compounds of the genus disclosed herein, other compounds can be discovered that are capable of modulating calcium ion channel activity, in particular those channels related to the RyR series of calcium ion channels.

Provided herein is a highly-efficient assay for high-throughput screening of other compounds that are capable of modulating calcium ion channel activity. By the way of example, and as shown in Example 4 below, a highly-efficient assay for high-throughput screening for small molecules is developed by immobilizing FKBP, either FKBP12 or FKBP12.6 (e.g., wild-type FKBP12.6 or a fusion protein, such as GST-FKBP12.6) onto a 96-well plate coated with glutathione, using standard procedures. PKA-phosphorylated ryanodine receptor (RyR), specifically RyR1 or RyR3 in the case of FKBP12 and RyR2 in the case of FKBP12.6, is loaded onto the FKBP-coated plate, and incubated with compounds at various concentrations (10-100 nM) for 30 min. Thereafter, the plate is washed to remove the unbound RyR, and then incubated with anti-RyR antibody (e.g., for 30 min). The plate is washed again to remove unbound anti-RyR antibody, and then treated with fluorescent-labeled secondary antibody. The plate is read by an automatic fluorescent plate reader for binding activity.

Alternatively, RyR is PKA-phosphorylated in the presence of ³²P-ATP. Radioactive PKA-phosphorylated RyR is loaded onto an FKBP-coated, 96-well plate, in the presence of JTV-519 analogues and other compounds at various concentrations (10-100 nM) for 30 min. The plate is washed to remove the unbound radiolabeled RyR, and then read by an automatic plate reader. PKA-phosphorylated RyR also is coated to the plate, and incubated with ³²S-labeled FKBP in the presence of the compounds.

### EXAMPLES

The present invention is described in the following Examples, which are set forth to aid in the understanding of the invention, and should not be construed to limit in any way the scope of the invention as defined in the claims which follow thereafter.

In particular, although the following Examples are directed to RyR2, other RyRs, namely RyR1 and RyR3, behave essentially in the same manner. Additional details can be found in U.S. Pat. No. 7,312,044, the contents of which are hereby incorporated by reference thereto.

### Example 1

### Expression of Wild-Type RyR2 and RyR2-S2809D Mutants

Mutagenesis of the PKA target site on RyR2 (RyR2-S2809D) was performed, as previously described (Wehrens et al., FKBP12.6 deficiency and defective calcium release channel (ryanodine receptor) function linked to exercise-induced sudden cardiac death. Cell, 113:829-40, 2003). HEK293 cells were co-transfected with 20 μg of RyR2 wild-type (WT) or mutant cDNA, and with 5 μg of FKBP12.6 cDNA, using Ca²⁺ phosphate precipitation. Vesicles containing RyR2 channels were prepared, as previously described (Wehrens et al., FKBP12.6 deficiency and defective calcium release channel (ryanodine receptor) function linked to exercise-induced sudden cardiac death. Cell, 113:829-40, 2003).

### Example 2

### RyR2 PKA Phosphorylation and FKBP12.6 Binding

Cardiac SR membranes were prepared, as previously described (Marx et al., PKA phosphorylation dissociates FKBP12.6 from the calcium release channel (ryanodine receptor): defective regulation in failing hearts. Cell, 101:365-76, 2000; Kaftan et al., Effects of rapamycin on ryanodine receptor/Ca²⁺-release channels from cardiac muscle. Circ. Res., 78:990-97, 1996). ³⁵S-labelled FKBP12.6 was generated using the TNT™ Quick Coupled Transcription/Translation system from Promega (Madison, Wis.). [³H] ryanodine binding was used to quantify RyR2 levels. 100 μg of microsomes were diluted in 100 μl of 10-mM imidazole buffer (pH 6.8), incubated with 250-nM (final concentration) [³⁵S]-FKBP12.6 at 37° C. for 60 min, then quenched with 500 μl of ice-cold imidazole buffer. Samples were centrifuged at 100,000 g for 10 min, and washed three times in imidazole buffer. The amount of bound [³⁵S]-FKBP12.6 was determined by liquid scintillation counting of the pellet.

### Example 3

### Immunoblots

Immunoblotting of microsomes (50 μg) was performed as described, with anti-FKBP12/12.6 (1:1,000), anti-RyR-5029 (1:3,000) (Jayaraman et al., FK506 binding protein associated with the calcium release channel (ryanodine receptor). J. Biol. Chem., 267:9474-77, 1992), or anti-phosphoRyR2-P2809 (1:5,000) for 1 h at room temperature (Reiken et al., Beta-blockers restore calcium release channel function and improve cardiac muscle performance in human heart failure. Circulation, 107:2459-66, 2003). The P2809-phosphoepitope-specific anti-RyR2 antibody is an affinity-purified polyclonal rabbit antibody, custom-made by Zymed Laboratories (San Francisco, Calif.) using the peptide, CRTRR1-(pS)-QTSQ, which corresponds to RyR2 PKA-phosphorylated at Ser²⁸⁰⁹. After incubation with HRP-labeled anti-rabbit IgG (1:5,000 dilution; Transduction Laboratories, Lexington, Ky.), the blots were developed using ECL (Amersham Pharmacia, Piscataway, N.J.).

### Example 4

### High-Throughput Screening Method

A highly-efficient assay for high-throughput screening for biologically-active small molecules is developed by immobilization of FKBP12.6 (GST-FKBP12.6 fusion protein) onto a 96-well plate coated with glutathione. PKA-phosphorylated ryanodine receptor type 2 (RyR2) or a RyR2 mutant as described above, radiolabeled with ³H-ryanodine is loaded onto the FKBP12.6-coated plate, and incubated with or without test compound(s) at various concentrations (10-1,000 nM) for 30 to 120 min. Thereafter, the plate is washed to remove the unbound radiolabeled RyR2 and then read by an automatic plate reader for binding activity.

In an alternative embodiment, RyR2 is not radio-labeled and the enhanced binding of RyR2 and FKBP12.6 is detected by using anti-RyR2 antibody. After the unbound RyR2 is removed, the plate is then incubated with anti-RyR2 antibody for 30 min. Afterwards, the plate is again washed to remove unbound anti-RyR2 antibody, and then treated with florescent-labeled secondary antibody. The plate is read by an automatic fluorescent plate reader for binding activity.

In an alternative embodiment, FKBP12.6, for example GST-FKBP12.6 fusion protein, is immobilized on a solid support and loaded onto a column. PKA-phosphorylated RyR2 or a RyR mutant (e.g., RyR2-S2808D or RyR1-S2844D) radiolabeled with ³H-ryanodine is loaded onto the column with or without test compound(s) at various concentrations (10-1,000 nM). The amount of radiolabeled RyR2 eluted from the column is quantified.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. An in vitro method for identifying a chemical compound that directly enhances binding of an FKBP protein to its corresponding ryanodine receptor (“RyR”), the method comprising:
(a) exposing the RyR to the FKBP protein in the presence or absence of a candidate chemical compound in vitro; and
(b) determining the amount of the RyR bound to the FKBP protein in the presence and absence of the compound, wherein an increased amount of RyR bound to the FKBP protein in the presence of the compound indicates that the compound directly enhances binding of the FKBP protein to the RyR; thereby
(c) identifying the compound as a compound that directly enhances binding of the FKBP protein to the RyR.

2. The method of claim 1, wherein step (a) comprises:
(i) immobilizing one of the FKBP protein or the RyR onto a solid phase;
(ii) contacting the other of the FKBP protein or the RyR with the solid phase; and
(iii) incubating the FKBP protein and the RyR in the presence or absence of a candidate chemical compound in vitro.

3. The method of claim 2, wherein the FKBP protein is immobilized.

4. The method of claim 2, wherein the RyR is immobilized.

5. The method of claim 1, wherein the RyR is PKA-phosphorylated as a result of disease state or in vitro PKA phosphorylation.

6. The method of claim 1, wherein the RyR is a RyR mutant that has low affinity for its corresponding FKBP protein.

7. The method of claim 6, wherein the RyR mutant is selected from the group consisting of RyR2-S2808D and RyR1-S2844D.

8. The method of claim 2, wherein the solid phase is a plate, a bead, a column or a combination thereof.

9. The method of claim 8, wherein the solid phase is a multi-well plate for high throughput screening.

10. The method of claim 9, wherein a predetermined number of wells on the multi-well plate contain no candidate chemical compound, and wherein other wells on said multi-well plate each contain one candidate chemical compound.

11. The method of claim 10, wherein the amount of the RyR bound to the FKBP protein in each well is determined by an automated plate reader.

12. The method of claim 1, wherein the RyR is radio-labeled or labeled with a fluorescent label.

13. The method of claim 12, wherein the RyR is radio-labeled with ³H-ryanodine, ³⁵S, ³²P, ³H, ¹⁴C or ¹²⁵I.

14. The method of claim 3, wherein the RyR is radio-labeled or labeled with a fluorescent label.

15. The method of claim 1, wherein the RyR is RyR1 or RyR3 and the FKBP protein is FKBP12.

16. The method of claim 3, wherein the RyR is RyR1 or RyR3 and the FKBP protein is FKBP12.

17. The method of claim 3, wherein the RyR is RyR2 and the FKBP protein is FKBP12.6.

18. The method of claim 4, wherein the RyR is RyR1 or RyR3 and the FKBP protein is FKBP12.

19. The method of claim 1, wherein binding of the RyR and the FKBP protein is detected by using an RyR-binding agent.

20. The method of claim 19, wherein the RyR-binding agent is an anti-RyR antibody.
